(19) United States
(12) Patent Application Publication
(10) Pub. No.: US 2024/0099869 A1
(43) Pub. Date: Mar. 28, 2024

(54) LANDING ZONE MARKER FOR FLOW DIVERTING STENT

(71) Applicant: Deepin Technologies, LLC, San Jose, CA (US)

(72) Inventor: Jon Schabert, San Jose, CA (US)

(73) Assignee: Deepin Technologies, LLC, San Jose, CA (US)

(21) Appl. No.: 18/471,024

(22) Filed: Sep. 20, 2023

Related U.S. Application Data
(60) Provisional application No. 63/410,629, filed on Sep. 28, 2022.

(51) Int. Cl. A61F 2/966; A61F 2/844; A61F 2/90; A61F 2/92

(52) U.S. Cl. CPC A61F 2/966; A61F 2/844; A61F 2/90; A61F 2/92; A61F 2002/9511; A61F 2250/0098

(57) ABSTRACT

An endovascular system includes an implant and a delivery system configured to deliver and deploy the implant to a treatment site in a vessel. The implant is radially expandable and configured to be deployed in vessels with diameters ranging from a minimal diameter to a maximal diameter. The delivery system comprises a proximal landing zone indicator comprising a radiopaque marker having a length from a proximal end to a distal end of the radiopaque marker, wherein the proximal end of the radiopaque marker indicates a position of the proximal end of the implant if deployed at a treatment site in a vessel having the minimal diameter, and the distal end of the radiopaque marker indicates a position of the proximal end of the implant if deployed at a treatment site in a vessel having the maximal diameter. A method of delivering an expandable implant to a target site in a patient's vascular system is also disclosed.

### CROSS-REFERENCE TO RELATED APPLICATIONS

This application claims priority to U.S. provisional patent application No. 63/410,629 filed Sep. 28, 2022 entitled “Landing Zone Marker for Flow Diverting Stent,” the disclosure of which is hereby incorporated by reference in its entirety.

### TECHNICAL FIELD

This application relates generally to medical devices and methods of using medical devices. In particular, various embodiments of a delivery system including an implant landing zone indicator and method are described.

### BACKGROUND

Intravascular implants such as stents and blood flow diverters are known and have been used in treating various vascular disorders. In general, an intravascular implant is delivered to in an elongate collapsed configuration a treatment site using a delivery system to navigate through the vasculature of a patient. Once at the treatment site, the implant is released from the delivery system and deployed in an expanded configuration. The deployed expanded configuration of an implant generally is shorter in length than the collapsed configuration of the implant constrained in a delivery system.

Accurate deployment of an intravascular implant is important especially in treating vessels having bifurcations. For instance, carotid stents are often deployed across a bifurcation between the common carotid artery and the internal carotid artery. If a stent is improperly deployed, the stent would have mal-apposition with the vessel and an edge or end of the stent may extend into the curve or turn of a bifurcated vessel. The mal-apposition must be then corrected by shortening the stent to move the proximal end into a straight segment, which would add significant complexity requiring manipulation of the stent and/or introducing a second device to correct. As braided stents tend to become larger and longer, they are constructed to achieve greater braid angles in order to provide the stents with a smaller pore size, resulting in significant shortening of the stents from a delivery configuration to a deployed configuration, which makes even more difficult to predict the proximal landing location of the stents during deployment.

Therefore, there remains a general need for a system to deliver and deploy implants in the patients' vascular systems. It would be desirable to provide a delivery system which can indicate landing locations of an expandable implant to facilitate accurate deployment at a treatment site.

### SUMMARY

In one aspect, embodiments of the disclosure feature endovascular system. In general, an embodiment of the endovascular system comprises an implant and a delivery system configured to deliver and deploy the implant to a treatment site in a vessel. The implant is radially expandable and configured to be deployed in vessels with diameters ranging from a minimal diameter to a maximal diameter. The delivery system comprises a proximal landing zone indicator comprising a radiopaque marker having a length from a proximal end to a distal end of the radiopaque marker, wherein the proximal end of the radiopaque marker indicates a position of the proximal end of the implant if deployed at a treatment site in a vessel having the minimal diameter, and the distal end of the radiopaque marker indicates a position of the proximal end of the implant if deployed at a treatment site in a vessel having the maximal diameter.

In various embodiments of the aspect, the delivery system comprises a tubular member and a delivery wire with the implant being disposed between the tubular member and the delivery wire for delivery. The radiopaque marker of the proximal landing zone indicator is provided on the delivery wire. The delivery wire can be a catheter and the radiopaque marker can be in the form of a coil on the catheter of the delivery wire.

In various embodiments of the aspect, the implant may be configured to be deployed in vessels with diameters ranging from the minimal diameter of 5 mm to the maximal diameter of 10 mm.

In various embodiments of the aspect, the implant comprises a braided stent or braided flow diverter, and is configured to be deployed in a vessel adjacent to a bifurcation.

In various embodiments of the aspect, the delivery system comprises a tubular member and a delivery wire with the implant being disposed between the tubular member and the delivery wire for delivery, and the radiopaque marker is provided on the tubular member.

In various embodiments of the aspect, the implant comprises a braided stent or braided flow diverter.

In a further aspect, embodiments of the disclosure feature a method of delivering an expandable implant to a target site in a patient's vascular system. The method comprises the following steps. The diameter of a vessel to be treated is determined. Then, an endovascular system comprising a delivery system and an implant is provided. The implant has a distal end and a proximal end and is radially expandable, and is configured to be deployed in vessels with diameters ranging from a minimal diameter to a maximal diameter, wherein the minimal diameter is equal to or smaller than the diameter of the vessel to be treated, and the maximal diameter is equal to or greater than the diameter of the vessel to be treated. The delivery system comprises a proximal landing zone indicator comprising a radiopaque marker having a length from a proximal end to a distal end of the radiopaque marker, wherein the proximal end of the radiopaque marker indicates a position of the proximal end of the implant if deployed in a vessel having the minimal diameter, and the distal end of the radiopaque marker indicates a position of the proximal end of the implant if deployed in a vessel having the maximal diameter. At a next step, the endovascular system is introduced to the vessel to be treated. A landing location for the distal end of the implant in the vessel is determined before the implant is deployed. Then, a landing location for the proximal end of the implant in the vessel before the implant is deployed. The determining of the landing location for the proximal end of the implant comprises observing a position of the proximal end and/or of the distal end of the radiopaque marker of the proximal landing zone indicator in the vessel and determining the landing location for the proximal end of the implant based on the diameter of the vessel to be treated and the position of the proximal end and/or of the distal end of the radiopaque marker of the proximal landing zone indicator in the vessel. The implant is deployed if the landing location for the proximal end of the implant in the vessel is determined to be desirable.

In various embodiments of the aspect, the method may further comprise repositioning the endovascular system before deploying the implant if the landing location for the proximal end of the implant in the vessel is determined to be undesirable.

In various embodiments of the aspect, the determining of a diameter of a vessel to be treated comprises determining a diameter of a vessel adjacent to a bifurcation.

In various embodiments of the aspect, the delivery system comprises a tubular member and a delivery wire with the implant being disposed between the tubular member and the delivery wire for delivery, and the radiopaque marker is provided on the delivery wire. The delivery wire may comprise a catheter and the radiopaque marker may be in the form of a coil on the catheter of the delivery wire.

In various embodiments of the aspect, the implant comprises a braided stent or braided flow diverter.

In various embodiments of the aspect, the delivery system comprises a tubular member and a delivery wire with the implant being disposed between the tubular member and the delivery wire for delivery, and the radiopaque marker is provided on the tubular member.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a brief summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1 illustrates a cross-sectional view of an example endovascular system according to embodiments of the disclosure.

FIG. 2 illustrates a cross-sectional view of an example endovascular system according to alternative embodiments of the disclosure.

FIG. 3 is a flowchart illustrating an example method according to embodiments of the disclosure.

FIGS. 4A-4C illustrate a method of using an example endovascular system according to embodiments of the disclosure.

### DETAILED DESCRIPTION OF EMBODIMENTS

With reference to the figures, various embodiments of an endovascular system and method will be described. The figures are intended to facilitate illustration and are not necessarily drawn to scale. Certain specific details may be set forth in the figures and description to provide a thorough understanding of the disclosure. It will be apparent to one of ordinary skill in the art that some of these specific details may not be employed to practice embodiments of the disclosure. In other instances, structures, materials, components, systems, and/or operations often associated with intravascular procedures may not be shown or described in detail to avoid unnecessarily obscuring description of embodiments of the disclosure.

It should be pointed out that while some embodiments of the disclosure are shown and described in conjunction with a procedure for treating disorder in a carotid artery, the device, system, and method described herein can be configured to treat disorders in other vasculatures or body lumens such as cerebral and peripheral vasculatures. The term “stent” may be used interchangeably with the term “implant.”

Embodiments of the disclosure provide a delivery system including a proximal landing zone indicator for deploying a braided stent or a radially expandable implant in a coronary, peripheral or other vasculatures. The proximal landing zone indicator includes a radiopaque marker which can indicate the length of the stent when used in the stent's labeled diameters. The distal end of the marker indicates the length of the stent when deployed in the largest vessel the stent is indicated for use whereas the proximal end of the marker indicates the length of the stent when deployed in the smallest vessel the stent is indicated for use. While a distal landing spot of the stent can be readily determined or verified by using a distal radiopaque marker on the delivery system, the proximal landing zone indicator of the disclosure allows for easy and reliable prediction of the proximal landing spot of the stent. The distal end of the proximal landing zone indicator shows where the stent's proximal end will land at the maximal labeled-diameter vessel, and the proximal end of the proximal landing zone indicator shows where the stent's proximal end will land at the minimal labeled-diameter vessel. The proximal landing zone indicator allows the user to view where the stent's proximal end will land before deploying the stent's distal end. The delivery system of the disclosure helps solve one of the most difficult operations in deploying a braided stent such as a carotid stent, ensuring the proximal end of the stent to land in a straight segment of a vessel or not in the middle of a bifurcated vessel e.g., between the internal carotid artery (ICA) and external carotid artery (ECA). The delivery system of the disclosure reduces or eliminates the need for re-sheathing a stent or for introducing another device to re-position the stent or correct mal-apposition.

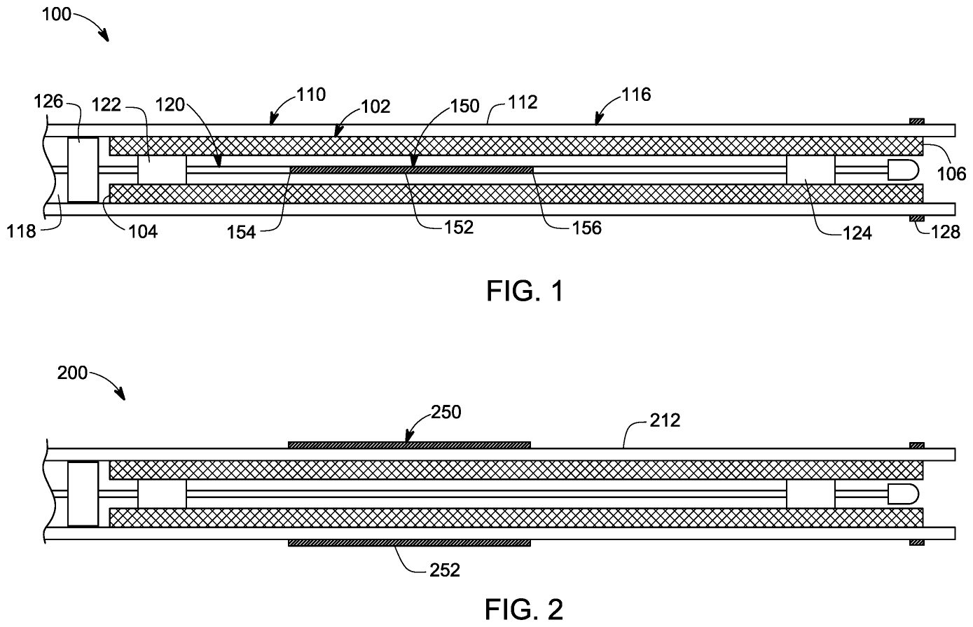

With reference to FIG. 1, an example endovascular system 100 according to embodiments of the disclosure is now described. In general, the endovascular system 100 comprises a tubular implant 102 and a delivery system 110 configured to deliver and deploy the implant 102 to a treatment site such as in a blood vessel or body lumen of a patient. The implant 102 comprises a proximal end 104 and a distal end 106 and is radially expandable. The implant 102 can be a resilient member, which can be compressed into a collapsed configuration for delivery and assume an expanded configuration upon release or deployment. The delivery system 110 includes a proximal landing zone indicator 150 to facilitate determination of a landing location for the proximal end of the implant 102 when deployed at a treatment site. The delivery system 110 may also include a distal marker 128 to indicate a landing location for the distal end of the implant at the treatment site.

With reference to FIG. 1, the implant 102 can be a stent, a blood flow diverter, or any other embolic implant, occlusion device for treatment of disorders in a vasculature or body lumen. By way of example, a stent or a blood flow diverter may include a mesh body having pores with a particular pore size. An example carotid stent may have a braided mesh body with a pore size ranging from 20 to 500 nanometers. The implant may also include a non-porous, non-permeable biocompatible material, cover or the like.

The implant 102 can be constructed from various shape-memory materials including metallic materials, polymeric materials, or a combination of metallic and polymeric materials. Suitable metallic shape-memory materials include but are not limited to alloys of nickel-titanium (NiTi) or Nitinol®, CuZnAl, FeNiAl, or the like. Suitable polymeric shape-memory materials include but are not limited to polytetrafluoroethylene (PTFE), polylactide (PLA), ethylene-vinyl acetate (EVA), or the like.

The implant 102 can be formed in various ways. For example, the implant 102 may comprise a mesh body including plural strands, wires, filaments, or ribbons of a suitable material that are braided, or woven, or otherwise formed into a desired pattern or form. The implant 102 may also be formed by cutting a pattern from a tube or etching a pattern from a sheet of a suitable material. A sheet of a suitable material may be cut or etched to a desired pattern and then be rolled or otherwise formed into a tubular or other shape.

Because the implant 102 can be constructed of a flexible or elastic material or is radially expandable, the implant 102 can be deployed, or labeled for use, in vessels of a range of diameters from a minimal diameter to a maximal diameter. By way of example, the implant 102 can be configured to be used as a carotid stent and deployed at a treatment site in a carotid artery of a patient. Depending on the patients, a carotid artery may have an average diameter ranging from e.g., 5.0 mm to 7.0 mm. An expandable implant 102 can be constructed for deployment at a treatment site in the carotid artery having any of the following diameters: 5 mm, 5.5 mm, 6 mm, 6.5 mm, 7 mm, or any size therebetween. On the other hand, when an implant changes from a collapsed delivery configuration to an expanded deployed configuration, the length of the implant shortens. Depending on the diameter of the vessel where the implant is deployed, the extent of shortening of the implant varies. By way of example, a carotid stent labeled 7 mm×80 mm, or a carotid stent having a length of 80 mm in a vessel having a diameter of 7 mm, may be indicated for use or deployed in vessels having a range of diameters e.g., from 6.0 mm to 7.0 mm. In a collapsed configuration constrained in a delivery catheter, the carotid stent may have a length of 160 mm. If deployed at a treatment site in a vessel of a diameter of 7 mm, the stent will be 80 mm long in an expanded configuration. The same carotid stent will be 112 mm long in an expanded configuration if deployed in a vessel of a smaller diameter of 6.0 mm. Therefore, before deployment of the stent it would be necessary to determine the length the expanded stent would have, or the landing location of the proximal end of the stent, to avoid mal-apposition of the stent into a bifurcated vessel or ensure the stent is deployed in a straight segment.

With reference to FIG. 1, the delivery system 110 may comprise a tubular member 112 having a proximal end region (not shown in FIG. 1), a distal end region 116, and a working lumen 118 extending between the proximal end region and the distal end region 116. The tubular member 112 can be a sheath, catheter, or microcatheter. The tubular member 112 may have a dimension and size to reach locations in a vasculature such as a bifurcated blood vessel in the carotid artery or the like for deployment of the implant. The proximal end region or a portion of the proximal end region may remain outside of the patient and include components, structures, or features to allow the user to operate the delivery system. The tubular member 112 may have a variable degree of rigidity or flexibility along the length of the tubular member to facilitate push or navigate through a tortuous vasculature. The distal end region 116 may be dimensioned and/or sized to contain or constrain the expandable implant in the working lumen. The distal end region 116 of the tubular member 112 may be constructed or configured to be capable of axial movement relative to the implant 102. For example, the tubular member 112 may be retracted or proximally moved for deployment of the implant 102 at a treatment site. The tubular member 112 may include a lubricious inner liner such as a PTFE or ePTFE inner liner or a lubricious coating on the inner surface to facilitate relative movement between the tubular member 112 and the implant 102 for deployment and/or re-sheathing of the implant 102.

With reference to FIG. 1, the delivery system 110 may include a delivery wire 120 configured to facilitate deployment or re-sheathing of the implant 102. The delivery wire 120 may extend through the entire length of the working lumen 118 of the tubular member 112 as in e.g., an over-the-wire delivery system. Alternatively, the delivery wire 120 may extend through only a distal portion of the tubular member as in e.g., a rapid-exchange delivery system. The delivery wire 120 may be in the form of a catheter, a wire, or the like and can be constructed of any suitable material such as stainless steel, Nitinol, or other metallic materials. The delivery wire 120 may include an atraumatic end tip to avoid or reduce damaging the tissue during delivery and/or deployment of the implant 102. One or more pads 122, 124 may be coupled to the delivery wire 120 to facilitate deployment and/or re-sheathing of the implant 102. For example, the pad 122 may be constructed of an expandable material and can assert an outwardly radial force to the implant 102 against the tubular member 112. The friction force between the expandable pad 122 and the implant 102 constrained in the lumen 118 of the tubular member 112 may help retract or re-sheath the implant 102 into the tubular member 112 by moving the delivery wire 120 in a proximal direction. The friction force between the expandable pad 122 and the implant 102 may also help prevent proximal movement of the implant 102 when the tubular member 112 is withdrawn proximally for deployment of the implant 102. Alternatively, or additionally, a bumper or stoper 126 may be coupled to the delivery wire 120 to prevent the implant 102 from moving in a proximal direction, when e.g., the tubular member 102 is proximally moved relatively to the implant for deployment of the implant. The bumper or stoper 126 may be constructed of a soft material e.g., silicone or the like to protect the implant 102, e.g., to prevent the edges of the implant 102 from damage when the implant 102 is retracted or re-sheathed into the tubular member 112.

With reference to FIG. 1, the delivery system 110 may include one or more distal radiopaque markers 128 to indicate a landing location of the distal end 106 of the implant 102. The one or more distal markers 128 can be disposed on the tubular member 112 in close proximity to the open distal end of the tubular member 112. The distal markers 128 may overlay or align the distal end 106 of the implant 102 constrained in the lumen 118 of the tubular member 112, to indicate a landing location of the distal end 106 of the implant 102 when deployed. By way of example, as the tubular member 112 is withdrawn, or proximally moved relative to the implant 102 constrained in the lumen 118 of the tubular member 112, the implant 102 is prevented from moving proximally by the bumper or stoper 126, and/or by the friction force between the implant 102 and the expandable pad 122, 124. As the distal end tip of the tubular member 112 is withdrawn past the distal end 106 of the implant 102, the distal end 106 of the implant 102 starts to expand for deployment in the vessel. Therefore, the one or more distal markers 128 indicate the same or substantially same location as the distal end 106 of the implant 102 will be when deployed out of the tubular member 112. The user may use the distal markers 128 to select or determine a desired distal landing location for the implant 102 by observing the distal markers 128 e.g., on x-ray fluoroscope, and maneuvering the tubular member 112 until the distal marker 128 is positioned at a desired location. Suitable radiopaque materials for the distal markers 128 include but are not limited to platinum, gold, tungsten, tantalum, barium, lodin, bismuth, etc., or an alloy containing any of the above metals. The distal radiopaque marker 128 on the tubular member 112 may be in form of a band, a ring, or a coating.

With reference to FIG. 1, according to embodiments of the disclosure, the delivery system 110 includes a proximal landing zone indicator 150 for determining a landing location of the proximal end 104 of the implant 102 when deployed in a vessel. While the distal marker(s) 128 may be used to indicate the landing location for the distal end 106 of the implant 102, the proximal landing zone indicator 150 of the disclosure can be configured to indicate or predict the landing location of the proximal end 104 of the implant 102 when deployed in a vessel where the implant 102 may change from an elongate collapsed configuration to a shortened expanded configuration. The radiopaque marker 152 of the proximal landing zone indicator 150 can be constructed of a suitable radiopaque material which can be visualized e.g., via x-ray fluoroscopy. Suitable radiopaque materials for the distal markers include but are not limited to platinum, gold, tungsten, tantalum, barium, lodin, bismuth, etc., or an alloy containing any of the above metals. The radiopaque marker 152 may be in the form of a coil wound on the delivery wire 120. The radiopaque marker 152 may also be in the form of a band or coating on the delivery wire 120.

With reference still to FIG. 1, the radiopaque marker 152 of the proximal landing zone indicator 150 may include a proximal end 154 and a distal end 156. The proximal end 154 of the radiopaque marker 152 can be configured to indicate a position of the proximal end 154 of the implant 102 when the implant 102 is deployed in a vessel having a minimal diameter the implant 102 is indicated for use. The distal end 156 of the radiopaque marker 152 can be configured to indicate a position of the proximal end 104 of the implant 102 when the implant 102 is deployed in a vessel having a maximal diameter the implant 102 is indicated for use. By way of example, a carotid stent may be constructed and indicated for use in treatment of vessels having a range of diameters e.g., from the minimum of 5 mm to the maximum of 7 mm. The radiopaque marker 152 of the proximal landing zone indicator 150 can be configured so that the proximal end 154 of the radiopaque marker 152 indicates the landing location of the proximal end 104 of the stent 102 if deployed at a vessel of 5 mm, and the distal end 156 of the radiopaque marker 152 indicates the landing location of the proximal end 104 of the stent 102 if deployed at a vessel of 7 mm. The radiopaque marker 152 can therefore provide the user with a clear and precise indication with respect to the implant's proximal end 104 landing location when the implant 102 is deployed in a vessel having a diameter of 5 mm, or in a vessel of a diameter of 7 mm. If the implant 102 is to be deployed in a vessel having a diameter of e.g., 6 mm, the radiopaque marker 102 can also provide a clear and accurate indication when the implant 102 is deployed, i.e., adjacent to the middle between the proximal end 154 and the distal end 156 of the radiopaque marker 152. Indeed, the length of the radiopaque marker 152 can be configured to provide a clear and accurate indication for the landing location of the proximal end 104 of the implant 102 when deployed in a vessel of any diameter within the range of diameters the implant 102 is indicated for use. It should be noted that the above dimensions are provided for illustration only, and the present disclosure and claims are not so limited.

One of the advantages of the disclosure is that the delivery system 110 can provide an indication of landing location for the entire range of the length of a stent in an actual vessel the stent is being deployed. This allows for a much easier and more accurate determination of the proximal landing zone of the stent, solving one of the biggest issues in deployment of stents. Conventionally, software systems are used to estimate the lengths based on the images exported from the patient generally prior to the case being done. However, any changes in the patient's anatomy due to e.g., medications such as vasospasm suppressants, can alter the anatomy, making these tools less useful. Furthermore, conventional methods do not provide the user a real-time indication of the proximal landing zone. Conventional methods use a single marker for the stent length at the largest indicated diameter. As such, the user has to attempt to measure off that marker without any visual references of length to estimate the variation from that point in the landing zone, leading to errors in determining the actual landing location. By providing a direct visual reference of the entire range of lengths of the deployed stent from the minimal diameter to the maximal diameter the stent is indicated for use, the user does not need to guess where the landing zone is. The proximal landing zone indicator can provide the user a direct visual reference under x-ray imaging, enabling much more accurate predictions of the actual landing zone of the stent.

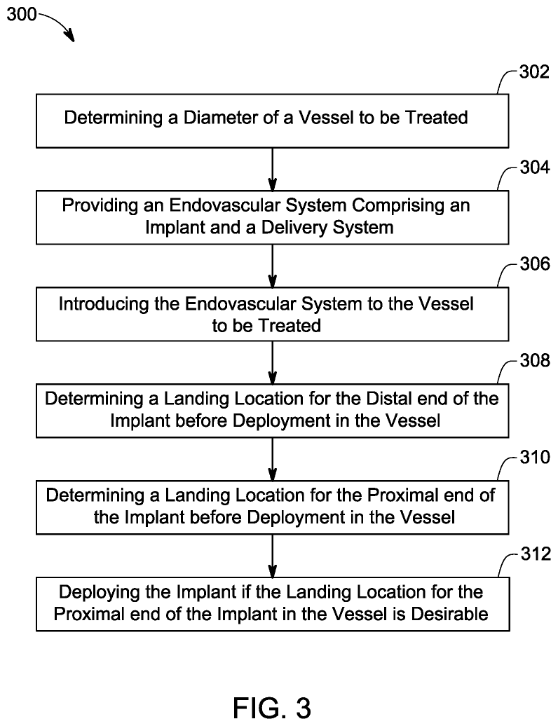

With reference to FIG. 2, an example endovascular system 200 according to embodiments of the disclosure is described. The endovascular system 200 shown in FIG. 2 is similar to the endovascular system 100 shown in FIG. 1 in many aspects, except that the proximal landing zone indicator 250 comprises a radiopaque marker 252 on the tubular member 212. The radiopaque marker 252 can be in the form of a coil wound on the tubular member 212, or a band, a ring, or a coating on the tubular member 212.

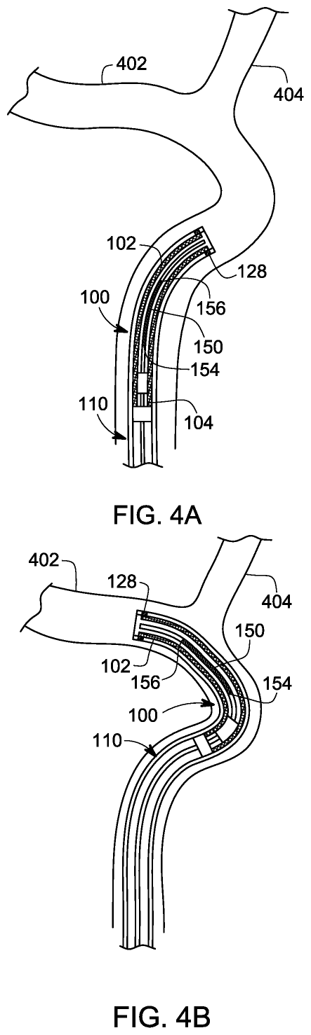

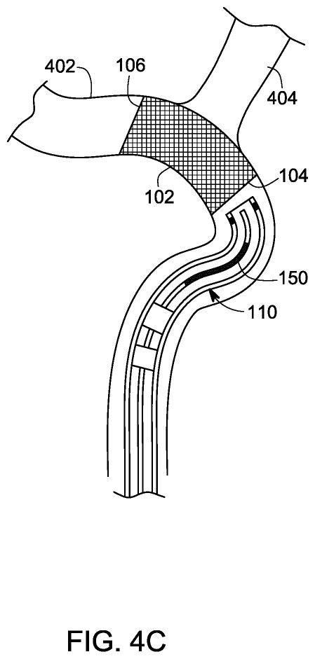

With reference to FIGS. 3 and 4A-4C, an example method 300 of delivering or deploying an implant to a treatment site is now described. The method 300 may start at step 302, where a diameter of a vessel 402 in a patient to be treated is determined. As described above, the actual length of a radially expandable implant at a treatment site is directly related to the diameter of the vessel where the implant is deployed. Furthermore, the vessel in a patient may change over time due to a variety of factors including e.g., temperatures, drugs taken, and so on. The diameter of a vessel in a patient to be treated can be determined by a variety of methods e.g., using microscopy, ultrasound, computed tomography (CT), magnetic resonance imaging (MRI), etc. The various techniques for measuring the diameter of a vessel are known and therefore their detailed description is omitted herein to focus on description of embodiments of the disclosure.

With reference to FIG. 3, at step 304 a delivery system containing an expandable implant is provided. The delivery system containing an expandable implant can be an endovascular system 100 described above in connection with FIG. 1 or an endovascular system 200 shown in FIG. 2. For example, the delivery system 110 may include a distal landing indicator 128 for indicating a landing location of the distal end of the implant 102 and a proximal landing zone indicator 150 for indicating a landing location of the proximal end of the implant 102. The proximal landing zone indicator 150 may include a radiopaque marker 152 having a proximal end 154 and a distal end 156, wherein the proximal end 154 of the radiopaque marker 152 is configured to indicate the position of the proximal end 104 of the implant 102 if deployed in a vessel having the minimal diameter the implant 102 is indicated for use, and the distal end 156 of the radiopaque marker 102 indicates the position of the proximal end 156 of the implant 102 if deployed in a vessel having the maximal diameter the implant 102 is indicated for use.

With reference still to FIG. 3, at step 306 the delivery system containing an implant is introduced to the vessel to be treated. For purpose of illustration, FIGS. 4A-4C shows a vessel 402 in the carotid artery of a patient having a bifurcation 404. The delivery system 110 may be introduced to the vessel 402 via an access e.g., in the femoral artery or groin area of the patient by using an introducer sheath. A guide catheter may be used to guide the endovascular system through the patient's vasculature. Fluoroscopy imaging may be used to monitor the endovascular system 100 in navigating through the patient's vasculature. The endovascular system 100 is advanced until the distal end of the implant 102 is positioned at or adjacent to a desirable distal landing location, as shown in FIG. 4A.

With reference to FIG. 3, at step 308 the landing location of the distal end of the implant in the vessel is determined or verified before the implant is deployed. This can be accomplished by observing the distal landing indicator or marker 128 on the tubular member of the delivery system 110 via x-ray fluoroscopy, as shown in FIG. 4B. If the landing location for the distal end of the implant 102 is determined to be undesirable, e.g., too close to or far away from the bifurcation 404, the endovascular system 100 may be further advanced or retracted, and the landing location for the distal end of the implant 102 is further determined or verified.

With reference still to FIG. 3, at step 310 the landing location for the proximal end of the implant 102 in the vessel 402 is determined or verified before the implant 102 is deployed (FIG. 4B). This can be accomplished by observing the proximal landing zone indicator 150 on the delivery system 110 via x-ray fluoroscopy, taking into account the diameter of the vessel 402 being treated as determined at step 302. By way of example, the delivery system 110 can be constructed to deliver a carotid stent 102 indicated for use in treatment of vessels having diameters ranging from the minimum of 5 mm to the maximum of 7 mm. The radiopaque marker 152 of the proximal landing zone indicator 150 can be configured so that the proximal end 154 of the radiopaque marker 152 indicates the landing location of the proximal end 104 of the stent 102 if deployed at a vessel of 5 mm, and the distal end 156 of the radiopaque marker 152 indicates the landing location of the proximal end 104 of the stent 102 if deployed at a vessel of 7 mm (FIG. 4C). Therefore, if the vessel 402 being treated has a diameter of 5 mm as determined at step 310, then the proximal end 104 of the stent 102, when deployed in the vessel 402, will be landed at a location indicated by the proximal end 154 of the radiopaque marker 152. If the vessel 402 being treated has a diameter of 7 mm as determined at step 302, then the proximal end 104 of the stent 102, when deployed in the vessel 402, will be landed at a location indicated by the distal end 156 of the radiopaque marker 152. If the vessel 402 being treated has a diameter of 6 mm as determined at step 302, then the proximal end 104 of the stent 102, when deployed in the vessel 402, will be landed at a location indicated by the middle of the radiopaque marker 152. In case that the landing location of the proximal end 104 of the stent 102 is indicated to be undesirable, e.g., too close to the bifurcation 404, the endovascular system 100 can be further advanced or retracted before deployment of the implant 102, and the landing location for the distal and proximal ends of the implant 102 is further determined.

With reference still to FIG. 3, at step 312 the user can release or deploy the implant if the landing location of the proximal end of the stent is indicated or determined to be desirable. As illustrated in FIG. 4C, the stent 102 may be released or deployed by proximally moving the delivery system 110 relative to the stent 102. During deployment, the stent 102 may be re-sheathed or retracted into the tubular member 112 by moving the delivery wire 120 in the proximal direction relative to the tubular member 112 (FIG. 1). Re-sheathing of the stent 102 may be needed in case the user determines that adjustment or repositioning of the implant's location is required. After the stent 102 is completely deployed, the delivery system 110 including the tubular member and the delivery wire may be withdrawn from the patient body.

Various embodiments of an aspiration catheter have been described with reference to figures. It should be noted that the figures are intended to facilitate illustration and some figures are not necessarily drawn to scale. Further, in the figures and description, specific details may be set forth in order to provide a thorough understanding of the disclosure. It will be apparent to one of ordinary skill in the art that some of these specific details may not be employed to practice embodiments of the disclosure. In other instances, well known components or process steps may not be shown or described in detail in order to avoid unnecessarily obscuring embodiments of the disclosure.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The term “proximal” and its grammatically equivalent refers to a position, direction or orientation towards the user or physician's side. The term “distal” and its grammatically equivalent refers to a position, direction or orientation away from the user or physician's side. The term “first” or “second” etc. may be used to distinguish one element from another in describing various similar elements. It should be noted the terms “first” and “second” as used herein include references to two or more than two. Further, the use of the term “first” or “second” should not be construed as in any particular order unless the context clearly dictates otherwise. All numeric values are provided for illustration and assumed to be modified by the term “about,” whether or not explicitly indicated. The term “about” generally refers to a range of numbers that one of skill in the art would consider equivalent to the recited value e.g., having the same function or result. The term “about” may include numbers that are rounded to the nearest significant figure. The recitation of a numerical range by endpoints includes all numbers within that range. For example, a range of 5 to 7 includes 5, 5.1, 5.2, 5.3, 5.4, 5.5, 5.6, 5.7, 5.8, 5.9, 6, 6.5, 7, and so forth.

Those skilled in the art will appreciate that various other modifications may be made. All these or other variations and modifications are contemplated by the inventors and within the scope of the invention.

What is claimed is:

1. An endovascular system, comprising:
an implant configured to be deployed in vessels with diameters ranging from a minimal diameter to a maximal diameter, the implant comprising a distal end and a proximal end and being radially expandable; and
a delivery system configured to deliver and deploy the implant to a treatment site in a vessel,
wherein the delivery system comprises a proximal landing zone indicator comprising a radiopaque marker having a length from a proximal end to a distal end of the radiopaque marker, wherein the proximal end of the radiopaque marker indicates a position of the proximal end of the implant if deployed at a treatment site in a vessel having the minimal diameter, and the distal end of the radiopaque marker indicates a position of the proximal end of the implant if deployed at a treatment site in a vessel having the maximal diameter.

2. The endovascular system of claim 1, wherein the delivery system comprises a tubular member and a delivery wire with the implant being disposed between the tubular member and the delivery wire for delivery, and wherein the radiopaque marker of the proximal landing zone indicator is provided on the delivery wire.

3. The endovascular system of claim 2, wherein the delivery wire comprises a catheter and the radiopaque marker is in the form of a coil on the catheter of the delivery wire.

4. The endovascular system of claim 2, wherein the implant is configured to be deployed in vessels with diameters ranging from the minimal diameter of 5 mm to the maximal diameter of 10 mm.

5. The endovascular system of claim 2, wherein the implant comprises a braided stent or braided flow diverter.

6. The endovascular system of claim 2, wherein the implant is configured to be deployed in a vessel adjacent to a bifurcation.

7. The endovascular system of claim 1, wherein the delivery system comprises a tubular member and a delivery wire with the implant being disposed between the tubular member and the delivery wire for delivery, and wherein the radiopaque marker is provided on the tubular member.

8. The endovascular system of claim 7, wherein the implant is configured to be deployed in vessels with diameters ranging from the minimal diameter of 5 mm to the maximal diameter of 10 mm.

9. The endovascular system of claim 7, wherein the implant comprises a braided stent or braided flow diverter.

10. The endovascular system of claim 7, wherein the implant is configured to be deployed in a vessel adjacent to a bifurcation.

11. A method, comprising:
determining a diameter of a vessel to be treated;
providing an endovascular system comprising a delivery system and an implant, wherein
the implant has a distal end and a proximal end and is radially expandable, the implant being configured to be deployed in vessels with diameters ranging from a minimal diameter to a maximal diameter, the minimal diameter being equal to or smaller than the diameter of the vessel to be treated, and the maximal diameter being equal to or greater than the diameter of the vessel to be treated, and
the delivery system comprises a proximal landing zone indicator comprising a radiopaque marker having a length from a proximal end to a distal end of the radiopaque marker, wherein the proximal end of the radiopaque marker indicates a position of the proximal end of the implant if deployed in a vessel having the minimal diameter, and the distal end of the radiopaque marker indicates a position of the proximal end of the implant if deployed in a vessel having the maximal diameter;
introducing the endovascular system to the vessel to be treated;
determining a landing location for the distal end of the implant in the vessel before the implant is deployed;
determining a landing location for the proximal end of the implant in the vessel before the implant is deployed, wherein the determining of the landing location for the proximal end of the implant comprises observing a position of the proximal end and/or of the distal end of the radiopaque marker of the proximal landing zone indicator in the vessel and determining the landing location for the proximal end of the implant based on the diameter of the vessel to be treated and the position of the proximal end and/or of the distal end of the radiopaque marker of the proximal landing zone indicator in the vessel; and
deploying the implant if the landing location for the proximal end of the implant in the vessel is determined to be desirable.

12. The method of claim 11, further comprising repositioning the endovascular system before deploying the implant if the landing location for the proximal end of the implant in the vessel is determined to be undesirable.

13. The method of claim 11, wherein the determining of a diameter of a vessel to be treated comprises determining a diameter of a vessel adjacent to a bifurcation.

14. The method of claim 11, wherein the delivery system comprises a tubular member and a delivery wire with the implant being disposed between the tubular member and the delivery wire for delivery, and wherein the radiopaque marker is provided on the delivery wire.

15. The method of claim 14, wherein the delivery wire comprises a catheter and the radiopaque marker is in the form of a coil on the catheter of the delivery wire.

16. The method of claim 14, wherein the implant comprises a braided stent or braided flow diverter.

17. The method of claim 11, wherein the delivery system comprises a tubular member and a delivery wire with the implant being disposed between the tubular member and the delivery wire for delivery, and wherein the radiopaque marker is provided on the tubular member.

18. The method of claim 17, wherein the implant comprises a braided stent or braided flow diverter.
